(19) United States
(12) Patent Application Publication
(10) Pub. No.: US 2024/0130997 A1
(43) Pub. Date: Apr. 25, 2024

(54) Compositions for ophthalmic care

(71) Applicant: ReBio Technologies Oy, Raisio (FI)

(72) Inventors: Harri Jukarainen, Raisio (FI); Jukka Tuominen, Raisio (FI); Jonathan Glen, Raisio (FI); Robert Watson, Raisio (FI)

(21) Appl. No.: 18/278,017

(22) PCT Filed: Feb. 21, 2022
(86) PCT No.: PCT/FI2022/050112

§ 371 Date: Aug. 20, 2023

(30) Foreign Application Priority Data
Feb. 19, 2021 (FI) 20215186

(51) Int. Cl. A61K 31/235; A61K 9/00; A61K 31/4045; A61K 31/4412; A61K 31/573

(52) U.S. Cl. CPC A61K 31/235; A61K 9/0048; A61K 31/4045; A61K 31/4412; A61K 31/573

(57) ABSTRACT

A controlled release pharmaceutical composition for eye injection for ophthalmic care. The composition comprises at least one active pharmaceutical ingredient and at least one biocompatible polymer and at least one biocompatible solvent. The composition, which is capable of forming an in-situ implant composition, comprises an injectable solution, suspension, emulsion or dispersion. The formulation can be injected through a needle that is suitable in size for an eye injection. The present compositions can be used for treatment of ophthalmic conditions, including glaucoma, dry and wet age-related macular degeneration, diabetic retinopathy, dry eye syndrome, and uveitis.

### FIELD OF INVENTION

The present invention relates to injectable long-term controlled release pharmaceutical compositions for ophthalmic care, their composition materials, formulations, manufacturing methods, application methods, and usage.

### BACKGROUND

Glaucoma is a progressive multifactorial disease characterised by damage to the optic nerve and progressive visual loss that, if left untreated, can lead to blindness. It is a significant cause of visual morbidity, accounting for falls, road traffic accidents, loss of independence and 12% of blind registrations. Open angle glaucoma (OAG) is the most common form, with a prevalence of about 2% among adults older than years; it is strongly associated with elevated intraocular pressure. Lowering intraocular pressure can slow progression of the disease and is the only treatment available. Raised intraocular pressure without optic nerve damage is termed ocular hypertension, which, in some patients, progresses to open angle glaucoma; lowering intraocular pressure reduces this risk.

The standard first-line treatment for OAG and ocular hypertension is eye drops that lower intraocular pressure, requiring multiple hospital visits for monitoring and treatment adjustment. Long-term and multiple topical medications are associated with multiple ocular and systemic side-effects, poor patient adherence, and are a risk factor for later surgical failure. Selective laser trabeculoplasty reduces intraocular pressure by increasing aqueous outflow through the trabecular meshwork with a single, painless outpatient laser procedure, minimal recovery time, and good safety profile. It was introduced in 1995 and received US FDA approval in 2001, yet is not routinely offered as first-line treatment. Selective laser trabeculoplasty superseded argon laser trabeculoplasty, with fewer adverse events, greater ease of use, and improved repeatability. The intraocular pressure-lowering effect is comparable to medical treatment and can delay or prevent the need for eye drops, avoiding the associated side-effects. The effect of selective laser trabeculoplasty is not permanent, but it can be repeated. When successful, it reduces the risk of non-adherence, by removing or lessening the need for complex treatment regimes. (www.thelancet.com Vol 393 Apr. 13, 2019) There is clear need to improve the outcome of the current medical OAG treatments as well as to avoid repeatable laser trabeculoplasty, i.e., an optimal patient friendly OAG treatment does not exist in the market.

Age-related macular degeneration (AMD) is one of the most common causes of irreversible blindness worldwide. In 2015 it affected 6.2 million people globally. In 2013 it was the fourth most common cause of blindness after cataracts, preterm birth, and glaucoma. It most commonly occurs in people over the age of fifty and in the United States is the most common cause of vision loss in this age group.

A minority of patients with early (dry) AMD can progress to the vision-threatening forms of AMD called late AMD. The commonest form of late AMD is “exudative” or “wet” AMD. Wet AMD occurs when abnormal blood vessels grow underneath the retina. These unhealthy vessels leak blood and fluid, which can prevent the retina from working properly. Eventually the bleeding and scarring can lead to severe permanent loss of central vision, but the eye is not usually at risk of losing all vision (going ‘blind’) as the ability to see in the periphery remains. There is a rarer form of late AMD called geographic atrophy, where vision is lost through severe thinning or even loss of the macula tissue without any leaking blood vessels.

Wet AMD is typically treated with intravitreal injections (injections into the eye) using a medicine called ranibizumab (also known as Lucentis, the brand name). Ranibizumab is one of a group of anti-VEGF medicines which, when injected into the eye on a regular basis, can stop the abnormal blood vessels growing, leaking and bleeding under the retina. Most people with wet AMD need to have these injections several times a year. Laser treatment is also available for AMD but is not effective for most cases. There is currently no treatment for dry AMD. Clearly existing AMD products are not optimal and there is a need for them to be improved upon with better clinical outcomes.

Uveitis is the inflammation of the uvea, the pigmented layer that lies between the inner retina and the outer fibrous layer composed of the sclera and cornea. Uveitis usually affects people aged 20 to 59, but it can occur at any age, including in children. Men and women are affected equally. It is estimated that two to five in every 10,000 people will be affected by uveitis in the UK every year. Despite being an uncommon eye condition, uveitis is a leading cause of visual impairment in the UK. It is further estimated that the more serious types of uveitis are responsible for one in every 10 cases of visual impairment in the UK.

The main treatment of uveitis is steroid medication (corticosteroids) which can reduce inflammation inside the eye. Several different types of steroid medication may be used, depending on the type of uveitis you have. Eye drops are often used for uveitis affecting the front of the eye, whereas injections, tablets and capsules are more often used to treat uveitis affecting the middle and back of the eye. In some cases, other treatments may also be needed in addition to corticosteroids. These include eye drops to relieve pain or widen (dilate) the pupil, a type of medication called an immunosuppressant, and, rarely, surgery.

Clearly existing uveitis products are not optimal and there is a need for them to be improved upon with better clinical outcomes.

Controlled release pharmaceutical compositions are known and are understood to be compositions designed to maintain the therapeutic level of an active pharmaceutical ingredient (API) over a specified (and at times a particularly extended) period of time.

DURYSTA™ (bimatoprost intracameral, solid biodegradable sustained-release implant) is indicated for the reduction of intraocular pressure (10P) in patients with open angle glaucoma (OAG) or ocular hypertension (OHT), however it is known to have challenging implantation site at anterior chamber and short 12 weeks release time. Similar issues have been seen with other marketed solid biodegradable products, such as OZURDEX®, which utilizes NOVADUR®technology.

Other examples of existing controlled release pharmaceutical compositions are those that function as an in-situ forming implant (ISFI). One such prior art drug delivery system is the ATRIGEL® system, which includes a bioresorbable polyester, and can be used for both parenteral and site-specific drug delivery (Pandya. Y. et al., International Journal of Biopharmaceutics. 2014; 5(3): 208-213.). In the ATRIGEL® drug delivery system, the polymer component and the API are dissolved in a biocompatible solvent. This solution of polymer and API is then administered by injection, after which it solidifies in vivo upon contact with aqueous bodily fluids and forms a solid implant.

Such in-situ forming implants may result in targeted drug delivery to a particular area and achieve some level of sustained release. However, drug delivery systems such as these still suffer from a high initial burst effect. Another in-situ forming implant including a bioresorbable polyester where the burst effect may be improved is disclosed in WO2014001904, where the in-situ forming implant is administered by subcutaneous injection. In WO2019097262 (A1) it was discovered that a polyester that has a reduced metal content in comparison to the prior art achieves improved controlled release when the polyester is included in a pharmaceutical composition. it was thought that the residual metal catalyst triggers degradation of the polyester, and of the API of the pharmaceutical formulation, where the polyester/API degradation adversely impacts the release profile of the eventual controlled release formulation. It was shown that the particularly low metal catalyst content of the provides a reduction in the burst effect, reduced lag time, improved API stability, and thus prolonged sustained and steady release, when these polyesters are included in a controlled release pharmaceutical composition.

However, none of the above mentioned ISFI were designed for eye injections due to the technical issues mentioned above, very small injection size, long release time and unsolved toxicity issues of ISFI due to the sensitivity of the eye.

### SUMMARY OF THE INVENTION

It is an aim of the present invention to provide a controlled release pharmaceutical composition for eye injection for ophthalmic care.

The composition comprises at least one active pharmaceutical ingredient and at least one biocompatible polymer and at least one biocompatible solvent, wherein
i) the composition is in the form of an injectable solution, suspension, emulsion or dispersion,
ii) the composition is in the form of an in-situ forming implant composition, and
iii) the biocompatible polymer is bioresorbable.

It is another aim of the invention to provide a method of treating a subject in need thereof with a controlled release pharmaceutical composition for eye injection for ophthalmic care, comprising administering to said subject an injection of a composition of the present kind.

More specifically, the present invention is mainly characterized by what is stated in the characterizing parts of the independent claims.

Considerable advantages are obtained by the invention.

Surprisingly, the formulation can be injected through a needle that is suitable in size for an eye injection through reducing the viscosity of the polymer/API formulation with a biocompatible solvent. Additionally, it was found that this solvent may be chosen so that it has no adverse toxicological effects on rabbit eye which is known to be more sensitive than the human eye. Thus, it is also assumed that no adverse effects will happen in human eye.

It has also been found that even from the very small injections such as 10 μL, or even smaller, such as from 2 up to 10 μL, very long sustained release of active ingredients was achieved. The smaller the volume of vitreous injection the smaller is the acute pressure elevation inside the eye right after the injection, which is beneficial. Also, the smaller the injection, the smaller is the risk of having something in vitreous blocking the sight or pathway to retina.

With the present compositions sustained release over 6 months can be reached even from injection volumes of no more than 10 to 20 μL

In preferred embodiments, the compositions comprise polyesters which have a reduced metal content. Typically they also have controlled structure and very low residual monomer content. Such compositions provide improved controlled release and multi-month release time when the polyester is included in a controlled release pharmaceutical composition for eye injection.

The present compositions are in particular endotoxin free, non-toxic formulations providing a reduction in the burst effect, reduced lag time, improved API stability, and thus prolonged sustained and steady release, when these polyesters are included in a controlled release pharmaceutical composition for eye injection.

Surprisingly, the present formulations can be injected through a needle that is feasible in size for an eye injection through reducing the viscosity of the polymer/API formulation with a biocompatible solvent. Additionally, it was found that this solvent may be chosen so that it has no adverse toxicological effects.

Surprisingly it was also found that even from the very small injections such as 10 μL, or smaller, very long sustained release of active ingredients was achieved. The smaller the volume of vitreous injection the smaller is the acute pressure elevation inside the eye right after the injection, which is beneficial.

The present compositions can be used for treatment of ophthalmic conditions, including glaucoma, dry and wet age-related macular degeneration, diabetic retinopathy, dry eye syndrome, and uveitis.

By the present invention, the total consumption of active pharmaceutical ingredient is reduced; using conventional dropwise administration a considerable part is lost and a significant portion thereof ends up in the systemic blood circulation.

In addition, examples of eye diseases that can be treated with the present sustained drug delivery methods include the following: Anophthalmia and Microphthalmia, Bietti's Crystalline Dystrophy, Behcet's Disease, Chataract, Coloboma, Idiopathic Intracranial Hypertension, Retinitis Pigmentosa, Retinoblastoma, Stargardt Disease, and Usher Syndrome.

### BRIEF DESCRIPTION OF THE DRAWINGS

FIG. 1 shows the results of injection force measurements for plasebo PLGA5050 (13 kDa) 40%/DMSO 60%;

FIGS. 2A and 2B show schematically in vivo implant formation: FIG. 2A depicts Subcutaneous ISFI SiSu® injection in a rat SC tissue, and FIG. 2B depicts intravitreal ISFI SiSu® injection in rabbit vitreous;

FIGS. 3A and 3B are photographs showing implant formation in vitro by 10 μl injection of formulation into PP pouch immersed in PBS buffer;

FIG. 4 is a simplified drawing of premixed formulation

FIG. 5 is a simplified drawing of the formation of a formulation using preparative syringe mixing;

FIG. 6 is a simplified drawing of the formation of a formulation using an injection pen;

FIGS. 7A and 7B show the long-term release of latanoprost from an in vitro implant as a function of time, FIG. 7A depicting the cumulative release and FIG. 7B the daily release;

FIGS. 8A, 8B and 8C and 8D show a second and a third example of the release of latanoprost from an in vitro implant as a function of time, FIGS. 8A and 8B depicting the release from implants with no additive and FIGS. 8C and 8D the release of a composition with triethyl citrate as an additive;

FIG. 9 shows the burst reduction and daily release rate increase of latanoprost from a composition containing benzyl benzoate as an additive (20 and 10 μL injections);

FIG. 10 shows graphically hydrolysis of latanoprost in porcine vitreous adjusted to pH 7.4 and with non-adjusted pH;

FIGS. 11A and 11B show the release of regorafenib from an in vivo implant as a function of time, FIG. 11A depicting the cumulative release and FIG. 11B the daily release;

FIGS. 12A and 12B show the release, as a function of time, of regorafenib from in vitro PLGA90:10-implants containing different amounts of benzyl benzoate, FIG. 12A depicting the cumulative release and FIG. 12B the daily release;

FIGS. 13A and 13B show the release, as a function of time, of regorafenib from an in vitro PLGA60:40-implant containing benzylalcohol, FIG. 13A depicting the cumulative release and FIG. 13B the daily release;

FIGS. 14A and 14B show the release of sunitinib from an in vivo implant as a function of time, FIG. 14A depicting the cumulative release with 55% of DMSO in formulation and FIG. 14B 65% of DMSO;

FIGS. 15A and 15B; FIGS. 15B and B shows how triacetin amount has an effect on the duration and level of released drug, with higher triacetin amounts the duration is shorter, and release higher FIGS. 16A and 16B show the effect of benzyl benzoate on the release of sunitinib from an in vitro implant as a function of time, FIG. 16A depicting the cumulative release and FIG. 16B the daily release;

FIGS. 17A and 17B shows that with 6% benzyl benzoate the 1 d burst of sunitinib malate can be totally avoided from an in vitro implant, FIG. 17A depicting the cumulative release and FIG. 17B the daily release;

FIGS. 18A and 18B show the release of dexamethasone (200 μg loading) from an in vitro implant as a function of time, FIG. 18A depicting the cumulative release and FIG. 18B the daily release; FIG. 18C and FIG. 18D results with 1200 μg dexamethasone loading and changing GA amounts in PLGA polymer

FIGS. 19A and 19B show the effect of BB (benzyl benzoate) and BA (benzyl alcohol) on the release of dexamethasone from an in vitro implant as a function of time, FIG. 19A depicting the cumulative release with BB additive and FIG. 19B the cumulative release with BA;

FIG. 20 shows the effect of SAIB (sucrose acetate isobutyrate) on the cumulative release of dexamethasone from an in vitro implant as a function of time;

FIG. 21 shows the effect of solvent choice (NMP/DMSO) and BB amount on the release of dexamethasone from an in vitro implant as a function of time;

FIG. 22 shows the release of exenatide (a peptide) formulated with triacetin as solvent and additive from an in vitro implant as a function of time;

FIG. 23 show the release of bovine serum albumin (BSA) from an in vitro implant comprising PLGA at ratio of 50:50 as a function of time; FIG. 23A cumulative release and FIG. 23B the daily release FIG. 24 shows the release of insulin from an in vivo implant as a function of time;

FIG. 25 shows the release of risperidone (molecular weight 410 g/mol) from an implant formulation of two different injection volumes as a function of time;

FIG. 26 shows graphically the release of exenatide from two different polymer matrices the formulations otherwise being the same;

FIG. 27 comprises a series of photos showing opened ex vivo porcine eyes with injected implant using various amounts of biocompatible solvent;

FIG. 28 shows cumulative release of latanoprost in PBS and in porcine vitreous with same composition as a function of time. The latanoprost released in vitreous is quantified as latanoprost acid, as quite immediate latanoprost hydrolysis occurs in vitreous;

FIGS. 29A and 29B show a PLGA8515 42%/DMSO 58% placebo implant at 5 months' time point inside rabbit eye pictured after sacrifice; and

FIG. 30 shows the dynamic viscosity of a composition of PGLA polymer (60% by weight) in DMSO (40% by weight).

### DESCRIPTION OF EMBODIMENTS

### Definitions

1. Intravitreal injection
   is a procedure to place a medication directly into the space in the back of the eye called the vitreous cavity, which is filled with a viscous fluid called the vitreous humor.
2. Intracameral injection
   is an injection of a substance into the eye anterior chamber.
3. In-situ forming implant
   In situ forming implant drug delivery systems provide a means by which a controlled release depot can be physically inserted into a target site without the use of surgery. ISFI avoid the use of large needles or microsurgery and they are injected as low viscous solutions and transform in the body to a gel or solid depot. Different triggers can be used to stimulate this transformation: (1) in situ cross-linking, (2) in situ solidifying organogels, and (3) in situ phase separation.
4. Biocompatible polymer
   Polymer that does not produce toxic or harmful products and stimulate an immune response in biological systems. This is essential so that during the treatment, the material does not induce a rejection response
5. Biodegrabable & (Bio)resorbable polymer & polyester
   As used herein, the term “bioresorbable polymer” refers to a polymer that is capable of removal by cellular activity and/or dissolution in a biological system such as the human body or in vivo. Typically, the present bioresorbable polyesters degrade in vivo by hydrolysis of their ester backbone into non-toxic products
6. Monomer
   As used herein, the term “monomer” takes its usual definition in the art and so refers to a molecular compound that may chemically bind to another monomer to form a polymer. The one or more monomers that form the polyester polymer disclosed herein are understood to include all enantiomers, diastereomers, racemates and mixtures thereof of the monomers in question.
7. As used herein, the term “metal catalyst” refers to a metal substance that accelerates and/or initiates the polymerisation reaction and has been added to the polymerisation system by purpose. As such, this term covers elemental metals, inorganic metal compounds, metal salts, oxides, halides, hydroxides, carboxylates, organometallic compounds, metal complexes metallocenes.
8. Placebo
   As used herein placebo means drug product without active ingredient.
9. Viscosity
   As used herein, viscosity stands for dynamic viscosity measured at 25° C. using a rheometer and extrapolating shear rate to 0 5-1. Unless otherwise stated herein or clear from the context, any percentages referred to herein are expressed as percent by weight based on a total weight of the respective composition.

Unless otherwise stated, properties that have been experimentally measured or determined herein have been measured or determined at room temperature. Unless otherwise indicated, room temperature is 25° C.

Unless otherwise stated, properties that have been experimentally measured or determined herein have been measured or determined at atmospheric pressure.

It must also be noted that, as used in the specification and the appended claims, the singular forms “a,” “an” and “the” include plural referents unless otherwise specified.

As used herein, the term “about” refers to a value which is ±5% of the stated value.

As used herein, unless otherwise indicated, the term “high molecular weight”, in particular when used with reference to proteins, stands for molecules having a molecular weight of more than 1 kDa, in particular more than 2 kDa, such as more than 3 kDa or more than 4 kDa. A high molecular weight protein may have generally a molecular weight of 1 to 200 kDa, for example 2 to 100 kDa.

As used herein, unless otherwise indicated, the term “average molecular weight” refers to a number average molecular weight (also abbreviated “Mn”).

As used herein, the molecular weight has been measured by gel-permeation chromatography using polystyrene standards.

The skilled person in the art will appreciate that the polyester is an example of the suitable polymer for the bioresorbable controlled drug delivery composition for eye injection and is not excluding other type of bioresorbable polyesters i.e. polyester can be manufactured from other monomers and polymers than lactides, glycolide or caprolactone, if different properties are desired in the biodegradable controlled drug delivery formulation, which will be apparent to those of skill in the art.

According to preferred embodiment of the present invention the following bioresorbable polymers, copolymers and terpolymers may be used for controlled drug delivery composition: Polylactide (i.e. poly(lactic acid), PLA), polyglycolide

(PGA) and poly(∈-caprolactone) (PCL), polydioxanone (PDO), polytrimethylenecarbonate (PTC), polylactides (PLA), poly-L-lactide (PLLA), poly-DL-lactide(PDLLA), PolyL-DL-lactide (PLDL), stereocopolymers; copolymers of glycolide, glycolide/trimethylene carbonate copolymers (PGA/TMC); other copolymers of PLA, such as lactide/tetramethylglycolide copolymers, lactide/trimethylene carbonate copolymers, lactide/d-valerolactone copolymers, lactide/∈-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/∈-caprolactone terpolymers, PLA/polyethylene oxide copolymers; polydepsipeptides; unsym metrically (3,6-substituted)-poly-1,4-dioxane-2,5-diones; polyhydroxy-alkanoates, such as polyhydroxybutyrates (PHB); PHB/b-hydroxyvalerate copolymers (PHB/PHV); poly-b-hydroxypropionate (PHPA); poly-p-dioxanone (PDS); poly-d-valerolactone-poly-∈-caprolactone, poly(∈-caprolactone-DL-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PVA); polypeptides; poly-b-malic acid (PMLA); poly-b-alkanoic acids; polycarbonates; polyorthoesters; polyphosphates; poly(ester anhydrides); and mixtures thereof; and natural polymers, such as sugars, starch, cellulose and cellulose derivatives, polysaccharides, collagen, chitosan, fibrin, hyaluronic acid, polypeptides and proteins.

In one embodiment, the polyester is formed from by polymerisation of lactide, glycolide, caprolactone, or combinations thereof. Such monomers are cyclic and are often used to form polymers by way of ring opening polymerisation. Preferably, the polyester is polylactic acid, polyglycolic acid, polycaprolactone, poly(lactide-co-glycolide), poly(lactide-co-caprolactone), or poly(glycolide-co-caprolactone), poly(lactide-co-glycolide-co-caprolactone, inclusive of all possible stereoisomers of these polymers.

Alternatively, the polyester can be formed by polymerisation lactic acid, glycolic acid and caprolactone or combinations thereof by condensation polymersation.

The polyester may be capped with polyethylene glycol (PEG), polypropylene glycol (PPG), and/or polyvinyl alcohol (PVA), at the terminus of one or more polymer chains. The polyester may also be a block co-polymer, comprising (in addition to one or more polyester blocks) at least one block of polyethylene glycol (PEG), polypropylene glycol (PPG), or polyvinyl alcohol (PVA).

The polyester can be linear or non-linear, such as branched (including star-shaped, hyperbranched and dendrimeric polymers).

In one embodiment, the polyester is non-linear, in particular branched, such as star-shaped. It has been found that using non-linear, in particular branched, such as star-shaped polyesters release of API is better controlled—in particular, the release rate of the API is more even.

Generally, in the present context, the term “branched polymer” stands for a polymer which has at least 3 terminal groups. In one embodiment, the branched polyester has 4 or more terminal groups, for example 4 or 6 terminal groups.

In embodiments, the use of branched polyesters can reduce viscosity of the injectable composition and, as a result, thin needles can be used for the injections.

The number average molecular weight of the polyester typically varies in the range of 1,000 g/mol to 250,000 g/mol, depending on the configuration and shape of the polyester molecule.

In some embodiments, the molecular weight of polylactides or lactide and glycolide copolymers is about 2,000 to 100,000 g/mol, such as 5,000 to 50,000 g/mol.

In one embodiment, the composition is formulated for use with an injection needle having a diameter corresponding to any of classes G20 to G31, for example G26 to G31.

The metal content of the purified polyester is >0 ppm and <40 ppm, and the total residual monomer content is preferably <0.2 wt %, in particular <0.1 wt %. Residual monomer content is measured using and GC or GC-MS against suitable monomer standards, which is a method familiar to those skilled in the art. Preferably, the total residual monomer content of the purified polyester may be <0.05 wt % or <0.01 wt %. The metal content of the purified polyester may be <35 ppm, <30 ppm, <25 ppm, <ppm, <15 ppm, <10 ppm, <5 ppm, or <2 ppm.

The ppm (i.e. parts per million) refers to the mass of elemental metal present in relation to the total mass of the polyester, e.g. a value of 1 ppm refers to 1×10-6 grams of elemental metal per 1 gram of polyester.

The metal content refers to the total amount of metal present in the polyester, inclusive of free metal, metal weakly coordinated to the polymer, plus metal strongly coordinated or bonded to the polymer. For example, the metal is selected from tin, zinc, iron, aluminium, titanium, platinum, bismuth, manganese, antimony, nickel, calcium, magnesium, sodium, lithium, yttrium, lanthanum, samarium, zirconium, ruthenium or combinations thereof but not excluding other typical polyester catalyst known in the art.

Polymers of the above kind are found to be especially suitable for controlled release pharmaceutical ISFI composition for eye injection:

Without wishing to be bound by theory, it is thought that these residual metal catalysts and residual monomers trigger degradation of the polyester, and also degradation of the API of the pharmaceutical formulation, where this polyester/API degradation adversely impacts product shelf life time, the release profile and time of the eventual controlled release formulation.

It is thought that the particularly low metal catalyst and residual monomer content of the polyesters disclosed herein provides a reduction in the burst effect, reduced lag time, improved API and polymer stability, and thus prolonged sustained and steady release, when these polyesters are included in a controlled release pharmaceutical composition for eye injection.

In an embodiment, the polyester is purified by the following method: i) the polyester is dissolved in an organic solvent wherein the organic solvent comprises at least one heteroatom selected from oxygen, nitrogen, sulphur, chlorine and phosphorus; ii) the metals are coordinated with organic solvent and other impurities such that residual monomers are separated from the polyester during the precipitation step and iii) in separation and washing step. It was surprisingly found that this results in a greater reduction in the metal content and residual monomer content in comparison to prior art purification methods.

Disclosed herein is a controlled release pharmaceutical composition for eye care injection comprising at least one active pharmaceutical ingredient, at least one biocompatible solvent, optionally one or more excipient(s)/additive(s) and at least one of the purified polyester disclosed herein.

The one or more active pharmaceutical ingredients comprised by the compositions disclosed herein are understood to include all enantiomers, diastereomers, racemates and mixtures thereof. The drug can also be a salt, a solvate, a hydrate, a pro-drug, a co-crystal, a derivative, in free base form, or a mixture thereof.

The pharmaceutical composition may also additionally include other safe excipients and ingredients for improving its properties/usability, such as formulation or API stability, drug release profile, and injectability.

Examples of excipients include biocompatible excipient, in particular selected from vanillin, triacetin, benzyl benzoate, benzyl alcohol, triethyl citrate, acetyl triethyl citrate, anisole, SAIB, hydroxypropylmethylcellulose, HP-b-CD, cyclodextrins, MCT (medium chain triglycerides), polycaprolactone diols, dextrans, sucrose, crown ethers, chitosan, mannitol, trehalose, or combinations thereof.

The active pharmaceutical ingredient comprised by the controlled release pharmaceutical compositions for eye injections disclosed herein may include one or more anti-VEGFs, VEGFs, Tyrosine kinase inhibitors, antiparasites, H2 receptor antagonists, antimuscarinics, prostaglandins and prostaglandin analogues, non-steroidal anti-inflammatory agents, proton pump inhibitors, aminosalycilates, corticosteroids, chelating agents, cardiac glycosides, phosphodiesterase inhibitors, thiazide, diuretics, anesthetic agents, carbonic anhydrase inhibitors, antihypertensives, anti-cancers, anti-depressants, calcium channel blockers, analgesics, opioid antagonists, antiplatels, anticoagulants, fibrinolytics, statins, adrenoceptor agonists, beta blockers, antihistamines, respiratory stimulants, micolytics, expectorants, barbiturates, anxiolytics, central nervous system agents, tricyclic antidepressants, 5HT1 antagonists, opiates, 5HT1 agonists, antiemetics, antiepileptics, dopaminergics, antibiotics, antifungals, anthelmintics, antivirals, antiprotozoals, antidiabetics, insulin and its derivatives, GLP-1 receptor agonists, thyrotoxins, female sex hormones, male sex hormones, antioestrogens, hypothalamics, pituitary hormones, posterior pituitary hormone antagonists, peptide drugs, protein drugs, protein kinases, antigens, antidiuretic hormone antagonists, bisphosphonates, dopamine receptor stimulants, androgens, steroid reductase inhibitors, non-steroidal anti-inflammatories, immunosuppressants, local anaesthetic, sedatives, anti-psoriatics, silver salts, topical antibacterials, vaccines, or vaccine antigens.

Preferably, the active pharmaceutical ingredient includes one or more tyrosine kinase inhibitors: 3-[4-(1-formylpiperazin-4-yl)-benzylidenyl]-2-indolinone, Acalabrutinib, Afatinib, Alectinib, Axitinib, Axotinib, Bosutinib, Brigatinib, Cabozantinib, Canertinib, Capmatinib, Cediranib, Ceritinib, Crenolanib, Crizotinib, Dabrafenib, Dacomitinib, Dasatinib, Dovitinib, Entrectinib, Erlotinib, Fedratinib, Flumatinib, Foretinib, Fostamatinib, Gefitinib, Geldanamycin, Genistein, Gilteritinib, Glesatinib, Ibrutinib, Icotinib, Imatinib mesylate, Lapatinib, Larotrectinib, Lestaurtinib, Lorlatinib, Lenvatinib, Midostaurin, Motesanib, Neratinib, Nilotinib, Nintedanib, Osimertinib, Pacritinib, Pazopanib, PD173955, Pexidartinib, Piceatannol, Ponatinib, Radicicol, Radotinib, Regorafenib, Ruxolitinib, Selpercatinib, Saracatinib, Savolitinib, Selumetinib, Sorafenib, Sunitinib, Tandutinib, Tesevatinib, Trametinib, Tucatinib, Vandertanib, Vatalanib, Vemurafenib.

More preferably, the active pharmaceutical ingredient includes one or more prostaglandins: Naturally occurring prostaglandins, Alprostadil, Bimatoprost, Carboprost, Cloprostenol, Dinoprostone, Enprostil, Fenprostalene, Fluprostenol, Iloprost, Latanoprost, Latanoprostene bunod, Luprostiol, Misoprostol, Netarsudil, Prostalene, Tafluprost, Travoprost, Unoprostone, Other preferred active pharmaceutical ingredients include dexamethasone and cyclosporine.

Most preferably, the active pharmaceutical ingredient includes one or more anti-VEGFs: Adalimumab, Aflibercept, Anecortave, Bevacizumab, Brolucizumab, Etanercept, Infliximab, Pegaptanib, Ranibizumab, Verteporfin and their biosimilars.

In one embodiment, the loading of the active pharmaceutical ingredient varies between 0.1 wt % and 90 wt % of the total weight of the polymer-solvent mixture, preferably 0.2 wt % to 50 wt %, more preferably 0.5 wt % to 30 wt %, most preferably from 1 wt % to 10 wt %.

Preferably, the controlled release pharmaceutical composition for eye injection is in the form of an in-situ forming implant composition. When the controlled release pharmaceutical composition for eye injection is in the form of an in-situ forming implant composition, the composition comprises at least one purified polyester disclosed herein and at least one active pharmaceutical ingredient and at least one biocompatible solvent and possibly additional excipients.

The term “biocompatible solvent” takes its usual definition in the art and so refers to a solvent that is not harmful or toxic to living tissue.

In one embodiment, the “biocompatible solvent” is miscible with water, in particular it is miscible with water in all proportions.

In one embodiment, the “biocompatible solvent” is further capable of dissolving, partially or wholly, the biodegradable polymer.

In one embodiment, the biocompatible solvent is a liquid capable of dissolving at least 1 mg/ml of the purified polyester at 35 to 37° C., and capable of dissolving, dispersing or suspending at least one active pharmaceutical ingredient.

In one embodiment, the biocompatible solvent is selected from N-methyl-2-pyrrolidone, triacetin, dimethylsulfoxide, anisole, benzyl benzoate, benzyl alcohol, acetone, butyl acetate, propyl acetate, ethyl acetate, methyl acetate, ethyl formate, isopropyl acetate, glycofurol or combinations thereof.

In one embodiment, the biocompatible solvent is selected from dimethylsulfoxide, optionally in combination with benzyl benzoate.

Typically, the weight ratio between biocompatible polymer and biocompatible solvent varies in the range from 1:99 to 99:1, preferably from 10:90 to 90:10, more preferably from 20:80 to 80:20, most preferably from 40:60 to 60:40.

In one embodiment, the injection volume varies between 1 microliter and 1000 microliter, preferably between 2 to 500 microliter. For example the injection volume can be in the range from 3 microliter to 100 microliter, more preferably from 5 microliter to 50 microliter, most preferably from 10 microliter to 30 microliter. For an intravitreally given injections volumes in the range from 5 to 25 μl are preferred, for subjconjunctivally given injections volumes in the range of 2 to 15 μl are preferred.

In one embodiment, the dynamic viscosity, at 25° C., of an injectable composition is between 10 and 100,000 mPas. Preferably the viscosity is 100 to 3000 mPas and more preferably 200 to 2000 mPas.

In one embodiment, the dynamic viscosity, at 25° C., of an injectable composition is about 800 to 1200 m Pas.

An embodiment comprises a method of treating a subject in need thereof with a controlled release pharmaceutical composition for eye injection, in any part of the eye, for ophthalmic care, or in therapy of ophthalmic conditions, comprising administering to said subject an injection of a composition of the present kind. In one embodiment, the injection is given to the eye intravitreally, intracamerally, periocularly, subchoroidally, subconjunctivally, subretinal, or under eye mucosal layer or utilizing pre-injected watery bleb under the surface of the eye. enable injection outside the sclera. Such a bleb will enhance the solidifying of the composition.

In one embodiment, an injection having a volume of 1 to 50 μl, in particular 2 to 25 μl, is given intravitreally or subconjunctivally

In one embodiment, the injection is given to the eye as needed or regularly between once a week and annually, in particular once each 3^{rd} to 6^{th} month.

The compositions of the invention are particularly useful in reducing intraocular pressure, especially intraocular pressure which is associated with glaucoma. The injectable ophthalmic compositions of the invention provide a steady release of an amount of API or combination of APIs which is effective in treating elevated intraocular pressure. The daily dosage and drug release duration is controlled by formulation composition and injection volume.

In embodiments of the present technology, drug release from the in situ implant will be effected through a succession of phases involving skin formation, swelling, shrinking, and degradation. In one embodiment, comprising an active ingredient (API), such as latanoprost, mixed with dimethyl sulfoxide (DMSO) as a primary solvent and benzyl benzoate (BB) as a cosolvent, a composition injected will, within minutes of injection, form a liquid implant surrounded by a thin skin, which will form a thicker shell, due to penetration of water into, and release of liquid components and API from, the injected composition. In a following stage, lasting up to a few weeks, further penetration of water and release, although now at a slower rate, of DMSO, BB and active ingredient, will give cause to swelling of the implant, solidification of the polymer and formation of a porous matrix. After about 2 weeks and lasting for the next 2 to 6 months or even longer, a solid implant, having a porous matrix, will release the active ingredient diffusing in the water phase present inside the porous solid implant from which it migrates into the biological surrounding. Gradually, the initially formed implant will degrade and the implant will start to shrink until it disappears due to degradation and bioresorption of the matrix. In one embodiment, the cosolvent is benzyl benzoate will be released at a slower rate than the active ingredient thus ensuring the beneficial effect of benzyl benzoate to the end of implant existence.

The following non-limiting examples illustrate the invention.

### EXAMPLES

Polymers

Biodegradable polyester polymers for ISFI formulations were made and purified using the methods described in patent application WO2019097262 (A1).

The polymers comprised polylactides (PLA) optionally with glycolic acid comonomers.

The ratio between the L- and D-isomers of the lactide monomers varied from 0 to 100 mole parts of L to 100 to 0 mole parts of D, typically a majority of the lactide monomers were L isomers, a polymer structure unit molar ratio of 100:0 to 70:30 (L-to-D) being particularly preferred.

The preferred polylactide homo- or copolymers (with glycolic acid) were branched and in one embodiment exhibited 4 terminal groups.

The following polylactides were employed:

PLGA5050, PLGA6040, PLGA4060, PLGA7030, PLGA8020, PLGA9010, PLA100, PLGA8515,

Preparation and Dissolution ISFI Formulations

Formulation Preparation and Dissolution Study

In one embodiment, the biodegradable polymer which is in powder form is dissolved in the biocompatible solvent in suitable size laboratory mixer. After the fluid is homogenous and free of particulates the desired active ingredient is added and the formulation is mixed again thus resulting in a homogenous formulation, either one phase clear fluid or dispersion or suspension. Sterile filtering can be done either before or after addition of active ingredient. If done after the addition of active ingredient, the formulation has to be free of solid active ingredient particles.

In one embodiment, the above mentioned formulation is loaded to a 100 μL glass syringe through suction from a vial when some air bubbles may arise to the syringe, then this loading is promptly emptied back to the formulation container, thus achieving bubble free syringe tip. Then the glass syringe is filled to 70 μL limit from formulation container, this way bubble free loading is achieved. The injection needle is attached to the syringe whilst pointing the syringe tip upwards, continuing with filling the needle until a drop can be seen at the end of the needle. The product is ready for injection.

In one embodiment, for dissolution study, the formulation is injected to the stainless steel or polypropylene mesh pouch (eye e.g. 210 μm). The pouch is settled in dissolution vial containing dissolution media (e.g. phosphate buffered saline at 35.5° C.) volume enough to remain sink conditions during dissolution study. At the wanted time points the pouch with the implant is transferred to a new dissolution vial with dissolution media and from the one remained the drug concentration is analysed through HPLC analysis. Thus cumulative and daily drug release are easily calculated.

Thumb Force

Thumb force should be measured to show the feasibility of the target product. The syringeability of the ISFI formulations were tested by practical thumb testing and Injection force measurements.

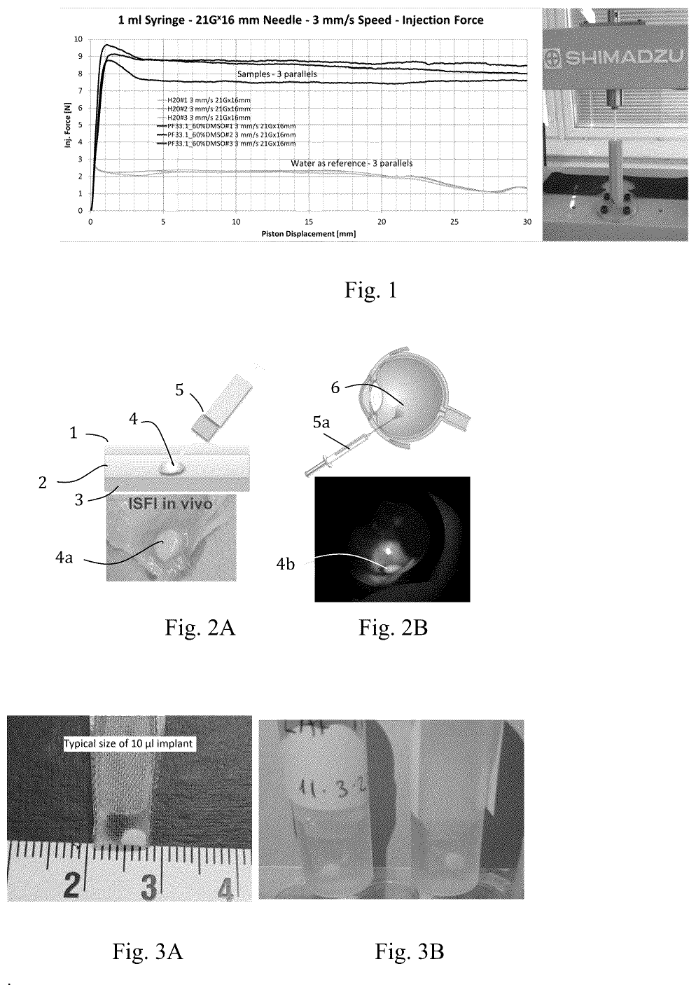

An example of a test is shown in FIG. 1 In the test setup, with 200 N cell, polyethylene 1 ml syringe, rubber seal in plunger, 21 G needle, L16 mm Water and air were used as references.

The results shown in the figure are at good thumb force level<10 N.

Implant Formation

Thinner Needles

Formation in vivo and in vitro of implant formulations were examined.

Different formulations were injected subcutaneously and intravitreally as illustrated in FIGS. 2A and 2B. In FIG. 2A, the reference numerals 1 to 3 are used for the epidermis 1, SC Fatty tissue 2 and the muscle 3. An in vivo implant 4 was formed by injection carried out with a syringe 5, 5a into an eye 6.

FIGS. 2A and 2B shows examples of in vivo implant formation by a) subcutaneous ISFI SiSu® injection 4A in a rat and b) intravitreal ISFI SiSu® injection 4B in rabbit.

FIGS. 3A and 3B shows examples of implant formation in vitro by 10 μl injection of formulation into PP pouch in PBS (pH 7.2) after 2 h incubation at 35.5° C. at 60 rpm using 26G needle, length 10 mm. The volume of the syringe used was 100p1.

Syringes

To illustrate the use of the final product, i.e. controlled release pharmaceutical composition for eye injection forming implant in situ, opening, mixing the product, needle attachment and injecting the product intravitreally, three examples are given, viz. Case 1: premixed formulation; Case 2: using preparative syringe mixing; Case 3: using pen.

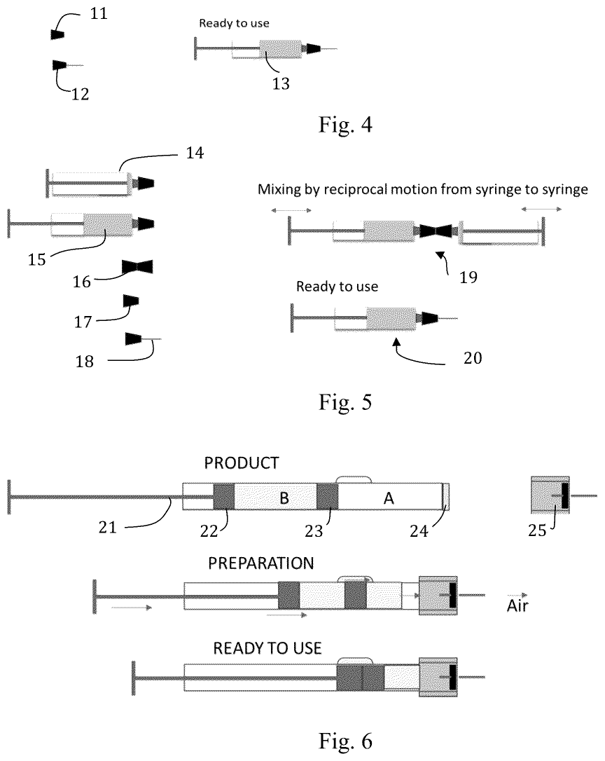

These cases are also shown the attached drawings: FIG. 4 illustrates Case 1, FIG. 5 illustrates Case 2, and FIG. 6 illustrates Case 3.

Case 1: Product must be at room temperature prior to injection. Final product is provided in the form of single sterile syringe 13 containing formulation.

Formulation comprises the mixture of drug, purified bioresorbable polyester and solvent in a fluid form. The syringe is capped. The cap 11 is removed from the syringe and user inserts hypodermic needle 12 to the tip. Prior to administration, syringe is purged in upright position from any large air bubbles through the needle. The dosage is injected to the desired place in the eye.

Case 2: This product must be at room temperature prior to injection. The final product is provided in the form of sterile part A and B in two syringes 19.

Part A comprises the weighed drug in the powder or liquid form in the tip of syringe A, 14 which is capped. Part B comprises the excipient purified bioresorbable polyester and solvent in a fluid form in another syringe B 15 which is also capped.

The caps are removed from the syringes and then they are combined with small sterile coupler 16 (one option is using syringes that can be coupled straight, e.g. syringes with male and female luer-lock). Mixing is carried out by compressing the fluid from syringe B through the coupler to the syringe A that has drug inside 19. Then the formed mixture of drug and fluid is compressed again from syringe A to the syringe B. This reciprocal movement is repeated 20 times and drug suspension, or solution is left to syringe A. The drug is then homogenously suspended or dissolved in the fluid in the syringe A 14. The syringe A is detached from coupler and a hypodermic needle 18 is inserted into the tip. Prior to administration, purge in upright position any large air bubbles from the needle. The dosage is then injected e.g. intravitreally to the desired place.

After injection, the needle is kept in its place approximately for few seconds before removing it from tissue.

Case 3: This product must be at room temperature prior to injection (cf. FIG. 6). The final product is provided in the form of pen that comprises two syringe pistons 22, 23, mandrel 21, septum 24, needle 25 with inner thread and transparent cylinder with side runner channel(s) holding pistons inside as depicted below.

The drug powder or liquid drug is stored in compartment A and purified bioresorbable polyester and solvent in a fluid form in compartment B separated by the pistons 23. Before administration following steps are carried out. The needle 25 is tightened to the septum 24 while the inner part of needle punctures the septum. Then the plunger 21 is pushed forward in upright position which makes the pistons 22 and 23 move with the fluid. Piston 23 stops moving when it reaches the position at the side runner channel(s) while piston 1 continues moving pushing the fluid from compartment B to compartment A through the side channel(s). Air from compartment A escapes from the needle. Mandrel is pushed forward until piston 22 reaches piston 23, when it stops. Now the drug and fluid are combined in compartment A. Next the drug is mixed with fluid by tapping it against other hand holding from cylinder until it is homogenous, tapping time depends on the tapping rate/speed.

Prior to administration, turn the mandrel clockwise 90° when further movement of the mandrel is allowed. Purge in upright position any air from the needle. Inject the dosage e.g intravitreally to the desired place in the eye. After injection keep the needle in its place approximately for few seconds before removing it from tissue.

In Vitro Release (Examples)

A number of active pharmaceutical ingredients (APIs), including latanoprost, regorafenib, sunitinib, dexamethasone, exenatide, BSA, insulin and risperidone, have been tested in vitro implant formulations for in vivo use.

### Example 1A

Latanoprost

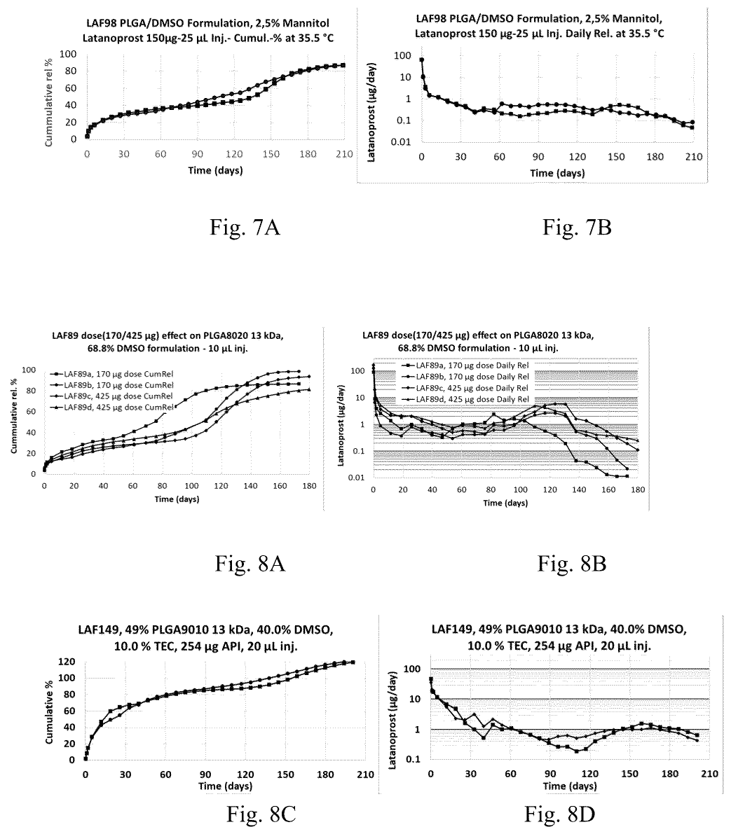

Latanoprost was tested for long-term drug release. The results are illustrated in FIGS. 7 to 9, depicting the long-term drug release on daily basis and cumulatively.

In vitro example of long term latanoprost release (35.5° C., PBS pH 7.2) containing 68% DMSO (LAF98) is depicted in FIGS. 7A and 7B.

FIGS. 8A, 8B and 8C, 8D show cumulative and daily release curves with two dosages. The effect of additives and excipients on the release of latanoprost was also tested (FIGS. 8C and D).

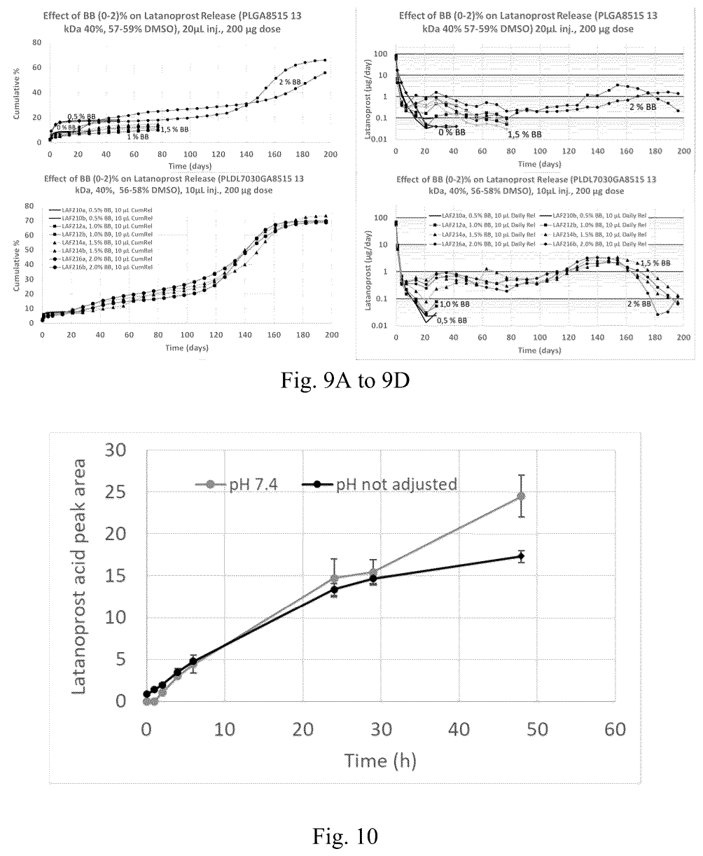

The effect of additive (benzyl benzoate, BB) shown in FIG. 9. As will appear, 2% of BB reduces the burst to 36% of the original value of formulation without the additive. The BB addition seems to reduce burst.

### Example 1B

Hydrolysis of latanoprost to latanoprost acid in porcine vitreous (cf. also dissolution tests within the vitreous)

Porcine vitreous was mixed with Hanks' balanced salt solution containing 50 mM Hepes (HBSS-buffer, pH 7.4) at and vitreous mix (200 μl) was pipetted to 96-well plates. The plates were incubated at +37° C. and 5% CO₂ with mild shaking for one hour to equilibrate the pH of vitreous into neutral range (pH 7.3-7.5). After incubation, 50 μl of 50 μg/mL latanoprost was added to plates and mixed. The final vitreous/buffer ratio was 1:1 and latanoprost concentration 10 μg/mL Latanoprost was incubated in vitreous at +37° C. and 5% CO₂ with mild shaking and samples were collected at time points (0, 1, 2, 4, 6, 24, 29, 48 hours). At each time point the 150 μl of the sample was mixed by vortexing with the 150 μl acetonitrile. The samples were stored at −20° C. until HPLC analysis.

Chemical stability of latanoprost was studied simultaneously in HBSS-buffer, pH 7.4, using the same protocol but replacing the vitreous with the buffer.

Hydrolysis of latanoprost was evaluated in buffer and in vitreous with pH adjusted to 7.4. No latanoprost acid was formed in mere buffer whereas in vitreous the amount of latanoprost acid was steadily increased during 48 h experiment (FIG. 10). This indicates that the hydrolysis of latanoprost in vitreous was mediated by enzymatic hydrolysis. Latanoprost acid formation in vitreous seemed to follow zero-order kinetics with the rate constant of 0.111 μg/hr.

### Example 2

Regorafenib

Regorafenib was used as an example API for tyrosine kinase inhibitor release from ISFI implant. Different formulations regorafenib ISFIs made using different PGLA polymers, solvents and additives using the methods described above chapters: All the ingredients were mixed together, and this homogenous mixture was loaded into the syringe with needle and injected to dissolution pouch. The release of the regorafenib was studied by taking samples from dissolution media and using UPLC for release calculations.

Solubility of regorafenib in water and PBS is negligible. Thus, Tween80 was added in PBS to increase solubility in the dissolution media. Regorafenib in vitro release studies from PGLA/DMSO ISFI were carried out in sink conditions, 2-5 ml PBS pH 7.2 (+Tween80 0.05%) in plastic and glass vessels at 35.5° C. (estimated at 10 mm depth in eye) and 60 rpm, orbit 61 mm in dark.

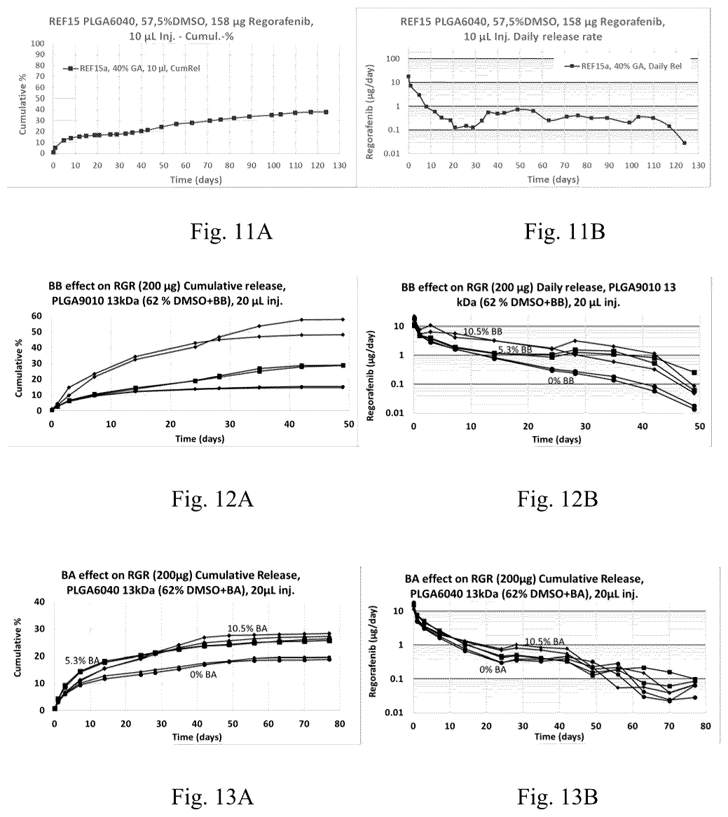

An example of effect of injection volume (10 and 20 μl) for regorafenib cumulative and daily release is shown in FIGS. 11A and 11612A for 4 months study.

An example of the effect of an additive (benzyl benzoate, BB) on regorafenib release is shown in FIGS. 12A to 12D.

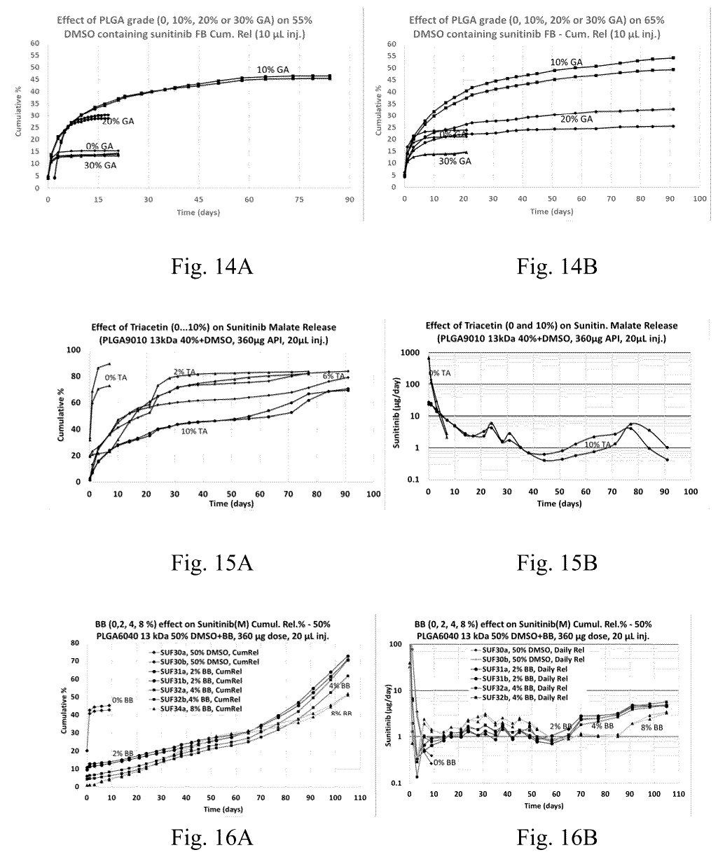

BA (benzyl alcohol) additive (0 . . . 10%) impact on regorafenib release. The results are shown in FIGS. 13A and 13B. These all are only examples and does not exclude other possible release rates.

### Example 3

Sunitinib

Sunitinib maleate and free base were used as example APIs for tyrosine kinase inhibitor release from ISFI implant. Different formulations sunitinib ISFIs made using different PGLA polymers, solvents and additives using the methods described above chapters: All the ingredients were mixed together, and this homogenous mixture was loaded into the syringe with needle and injected to dissolution pouch. The release of the sunitinib was studied by taking samples and using UPLC.

Sunitinib in vitro release studies from PGLA ISFI were carried out using sink conditions, 2-5 ml PBS pH 7.2 in plastic and glass vessels at 35.5° C. (estimated at 10 mm depth in eye) and 60 rpm, orbit 61 mm in dark. An example of effect of polymer composition (amount of glycolide in the polymer) for sunitinib free base cumulative and daily release is shown below where the formulation has been diluted with 55 or 65% of DMSO. There is a clear effect on both cases by which polymer grade have been used.

Release of sunitinib free base is shown in FIGS. 14A and 14B.

The effect of triacetin on the release rate of sunitinib malate is shown in FIGS. 15A and 15B.

An example of effect of additives on sunitinib release rate is shown in FIGS. 16A and 16B. In particular, the figures show the effect of BB (benzyl benzoate) addition in amounts of 0, 2, 4 and 8% on sunitinib release.

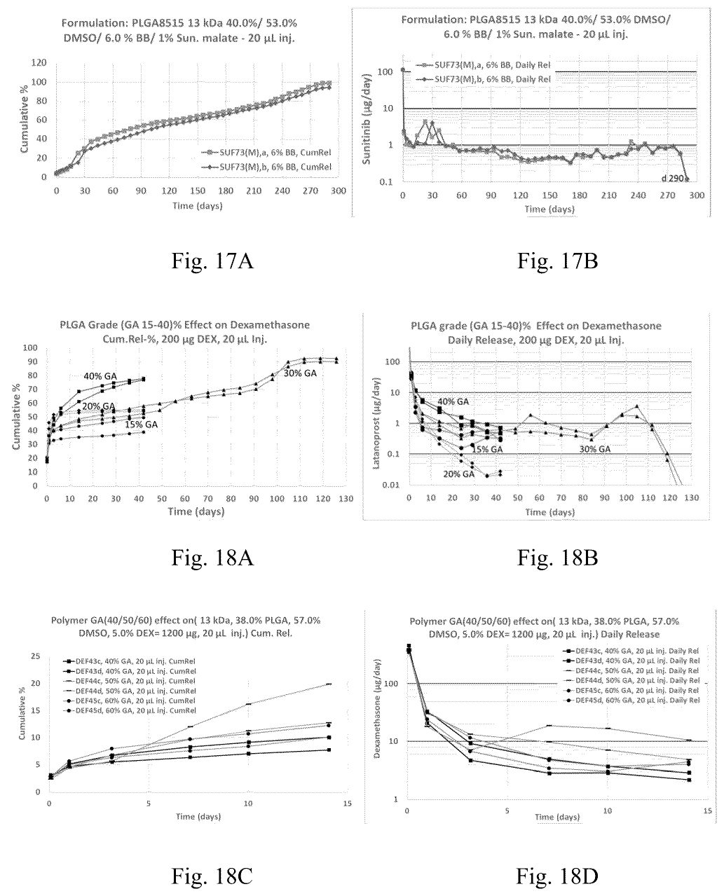

By use of additives, the burst can be eliminated totally. This was shown in one example employing 6% of BB as an additive and depicted in FIGS. 17A and 17B.

### Example 4

Dexamethasone

Dexamethasone was used as an example API for corticosteroid release from ISFI implant.

Different formulations for Dexamethasone ISFIs were made using different PGLA polymers, solvents and additives using the methods described above. Thus, all the ingredients were mixed together, and this homogenous mixture was loaded into the syringe with needle and injected to dissolution pouch.

The release of the Dexamethasone was studied by taking samples and analysing the samples with HPLC.

The effect of the glycolide portion in the PLGA polymer on the properties of the formulation are illustrated in FIGS. 18A and 18B, also in 18C and 18D with higher drug loading. It can be seen from the figures that the loading of the drug may have an effect on the burst behavior.

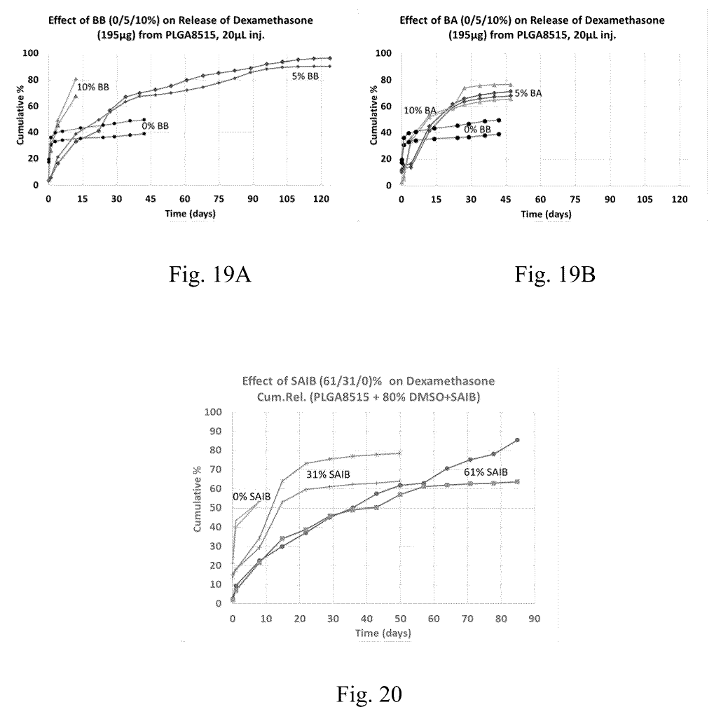

Dexamethasone in vitro release studies from PGLA ISFI were carried out, 2-5 ml PBS pH 7.2 in plastic and glass vessels at 35.5° C. (estimated at 10 mm depth in eye) and 60 rpm, orbit 61 mm in dark. An example of the effect of additive (benzyl benzoate, BB, and benzyl alcohol, BA) in formulation composition for Dexamethasone cumulative and daily release is shown in FIGS. 19A and 19B.

The effect of the of addition of SAIB to a formulation including PLGA which facilitates the solid implant formation is illustrated in FIG. 19 showing the cumulative effect as a function of time.

SAIB seems to reduce the burst for this formulation composition, where amount of polymer is constant.

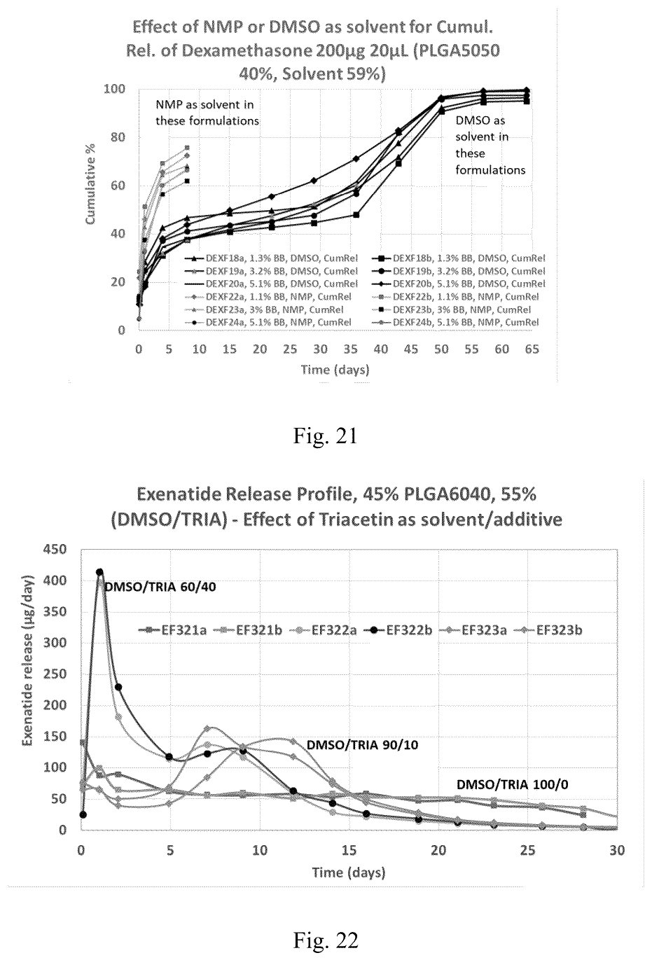

The effect of solvents (e.g. DMSO vs. NMP) and solvent mixtures were also studied. The example illustrated in FIG. 21 shows that NMP as a solvent gives higher burst for dexamethasone formulations with PLGA, which may also facilitate the drug release control through solvent selection or solvent blending.

### Example 5

Exenatide

Exenatide is an example of high molecular weight peptide. As an example for peptide (Exenatide) release below, the effect of additive/solvent (DMSO/triacetin) ratio for release profile, instead of pure solvents also peptide compatible additives may be applied if different drug release profiles are needed. The results are shown in FIG. 22.

### Example 6

BSA

BSA (bovine serum albumin) was tested as a model protein.

This example demonstrates the release of high molecular weight protein release from the ISFI formulation.

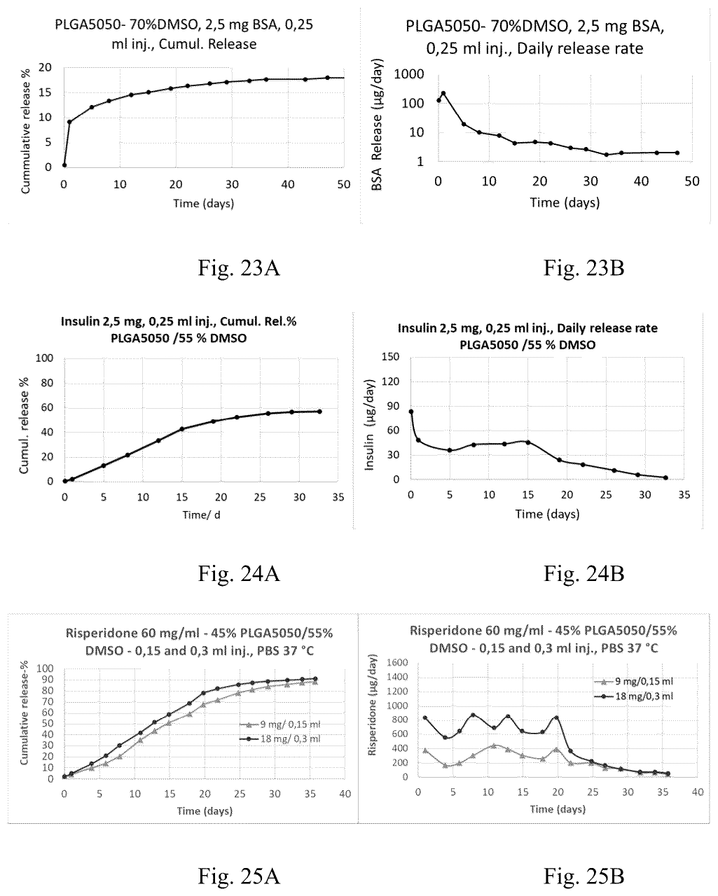

Purified PLGA5050 polymer was used for preparation of following formulation. PLGA5050 was dissolved in DMSO (70%) and subsequently powder bovine serum albumin (BSA, 10 mg/ml) was suspended in the polymer solution. The drug release rate was measured in phosphate buffered saline (PBS, pH 7.4) by injecting 0.25 ml of suspension in a stainless steel mesh screen pouch, and dropping the pouch in a PBS (12 ml) containing glass vessel, after which precipitation of the formulation started. The glass vessels were placed in a 37° C. tempered incubator with 60 rpm revolving speed (orbit 2 inches). The in vitro analysis of the BSA release was carried out by sampling the PBS and analysing the intact BSA in ultrahigh performance liquid chromatography equipment (UHPLC). After every sampling the whole PBS volume was replaced with fresh PBS. The cumulative and daily release profiles from the drug release experiment are shown in FIGS. 23A and 23B.

### Example 7

Insulin

The results for an example of insulin cumulative and daily releases are given in FIGS. 24A and 24B, respectively.

### Example 8

Risperidone

The effect of injection volume and thus also dose on small molecule release (risperidone, Formula C23H27FN4O2, molar mass 410 Daltons) are shown in FIGS. 25A and 25B; FIG. 25A depicting cumulative release and FIG. 25B depicting daily release.

### Example 9

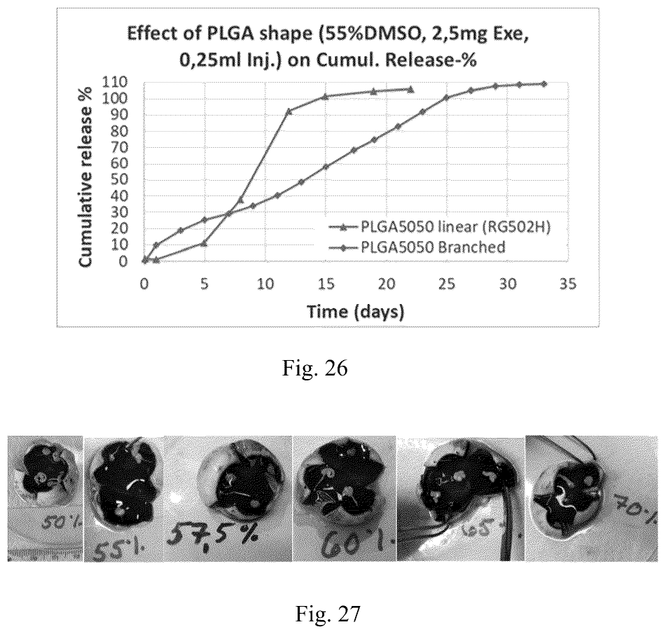

The influence of the polymer shape (linear vs. branched) of the resorbable biopolymer on the release of API is illustrated using a copolymer of polylactide and glycolic acid (PL:GA mole ratio of 50:50) in FIG. 26.

As will appear, with a branched polymer a steadier and longer drug release is achieved.

### Example 10

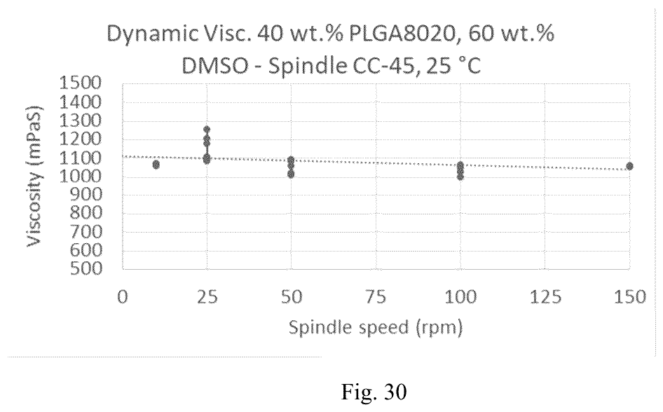

The dynamic viscosity at 25° C. of an injectable composition comprising PLGA (at a molar ratio of 80:20) in a solvent of DMSO is depicted as a function of spindle speed is shown in FIG. 30.

As will appear, the viscosity of this composition was in the range of about 900 to 1200 mPas.

Ex-Vivo and In-Vivo studies

Endotoxin test for the formulation, polymer and solvent Endotoxin tests were carried out for formulation: PLAG8515 42 wt. % and 58 wt. %

DMSO. Polymer was dissolved in DMSO (Gaylord, Procipient—USP, Ph. Eur) and then formulation was filter sterilized using 70% isopropanol and sterilized 100 μL (Luer Lock) syringes and 0.2 μm PTFE syringe filters inside disinfected (hydrogen peroxide+70% IPA wiping+UV 18 h) Class 5 isolator.

The microbiological bioburden and endotoxin tests for placebo formulation and drug (Latanoprost) containing formulation were carried out using LAL endotoxin kit.

Endotoxin tests showed that the studied formulation gave below 0.25 EU/ml values, which fulfills the criteria for eye applications.

Injection of ISFI into Isolated Pig Eye

The aim of this study was to clarify how different placebo formulations at different viscosities form an implant in porcine vitreous ex vivo.

Six Hamilton 100 μl LL syringes prefilled with placebo formulations at different DMSO (Gaylord, Procipient— USP, Ph.Eur) concentrations (50%, 55%, 57.5%, 60%, 65% and 70%), used polymer was PLGA8020. Six fresh porcine eyes (two months old female pigs; a′ ca 40 kg). 26G 10 mm needles. More details about the formulations in Table 1.

TABLE 1

| | | | | | Depth of |
|---|---|---|---|---|---|
| | | DMSO | Vol/ | | injection/ |
| Formulation | Polymer | wt. % | μL | Injections | mm |
| REB50% | PLGA8020 | 50 | 100 | 6 × 10 μL | 2, 3, 4, |
| | | | | | 5, 6, 7 |
| REB55% | PLGA8020 | 55 | 100 | 6 × 10 μL | 2, 4, 7 |
| REB57.5% | PLGA8020 | 57.5 | 100 | 6 × 10 μL | 2, 4, 7 |
| REB60% | PLGA8020 | 60 | 100 | 6 × 10 μL | 2, 4, 7 |
| REB65% | PLGA8020 | 65 | 100 | 6 × 10 μL | 2, 4, 7 |
| REB70% | PLGA8020 | 70 | 100 | 6 × 10 μL | 2, 4, 7 |

Injections: The needle in the syringe was inserted about 4.5 mm out of limbus at a 90° angle, through the sclera into the vitreous and the vehicle was slowly (ca. 5 s) injected into the vitreous.

The first injections were performed six times with 50% formulation at different depths into one eye (Table 1). The other five eyes were injected three times; 2, 4 and 7 mm. The depths were marked in the needles with a permanent marker.

Five minutes after the last injection, the cornea, iris and lens were removed from the eyeball to see vitreous and implants.

Results: There were no problems in the preparation of syringes and needles for the injections. Three depths (2 mm, 4 mm and 7 mm) were used after the first injection and formulation. The injection depths of between 4 and 5 mm were the best ones, as it is far enough from the retina and may not disturb an animal's sight. The 57.5% DMSO containing formulation showed to be the most suitable for intravitreal injections in porcine due to the implant shape and suitable thumb force needed for the injection. It is clear that the viscosity and thus implant formation is also effected by the molecular weight and composition of the polymer and the solvent type.

FIG. 27 shows photos of ex vivo porcine eyes with injected implants and with used solvent (DMSO) amount.

Dissolution Tests within the Vitreous

These tests were carried out to ensure that the latanoprost released from the implant would go through hydrolysis to latanoprost acid, which is the active form of the molecule. Purpose of this study was also study the conversion rate of latanoprost to latanoprost acid. In addition, the release of latanoprost from ReBio test formulations was evaluated in vitreous vs. in PBS.

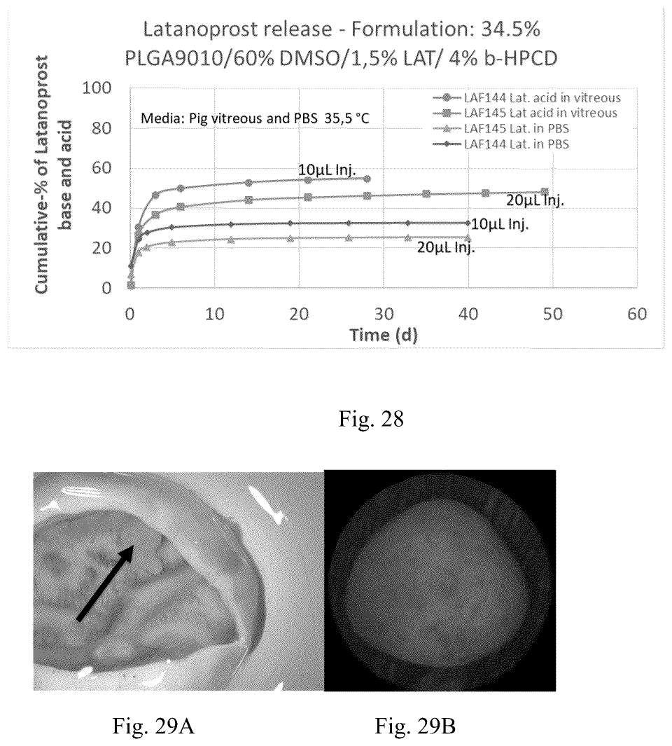

First hydrolysis of latanoprost was evaluated in buffer and in vitreous with pH adjusted to 7.4 and non pH adjusted. No latanoprost acid was formed in mere buffer whereas in vitreous the amount of latanoprost acid was steadily increased during 48 h experiment (FIG. 28).

This indicates that the hydrolysis of latanoprost in vitreous was mediated by enzymatic hydrolysis. Latanoprost acid formation in vitreous seemed to follow zero-order kinetics with the rate constant of 0.111 μg/hr.

Next the drug release from implant was also tested in porcine vitreous vs. PBS(=phosphate buffered saline). It was noticed as expected that the latanoprost did not change to its acid form in the PBS, thus below the cumulative drug release data is measured for latanoprost (LA) acid form in vitreous and ester form in PBS media. The release curves resemble each other, but the release is a bit higher in the vitreous, this is most probably due to the higher water solubility of the hydrolysed acid form of the latanoprost, and thus faster distribution in the media.

As discussed above, FIG. 10 shows the cumulative release of latanoprost and latanoprost acid from injected composition as a function of time in vitreous and PBS media at 35.5° C. Formulation composition in this example was 35% PLGA9010/60% DMSO/4% b-HPCD—180 μg Latanoprost.

Tolerability Studies in Rats and Rabbits

In order to test the tolerability of DMSO in eye, DMSO (USP, Ph. Eur.) was injected to the rat eyes intravitreally using 34 G needle The injected volume was 3 μl and three concentrations were injected; 60%, 1/10 dilution, 1/100 dilution. Used Ph. Eur. grade DMSO was 0.2 μm Filter sterilized in Class 5 isolator before the injection.

The rats were monitored for two weeks using the following endpoints:
fundus imaging, including blood vessels
cloudiness and other symptoms in the anterior part of the eye
optic nerve head
state of the vasculature in the posterior eye segment (FAG)
ERG
intraocular pressure
retinal thickness with OCT
histology (in the end)

Results: No adverse effect were noticed that could be related to DMSO in this study.

To study tolerability of placebo formulations, they were injected to the living rabbits' eyes (5 Rabbits, 10 eyes) intravitreally using 26G needle. Tested formulation was filter sterilized by filtering the formulation with 0.2 μm PTFE syringe filter before loading in the isolator to 100 μL syringes. The injected volume was 10 μL The formulation consisted of PLGA8515 42% and DMSO 58%, forming homogenous clear fluid.

The rabbits were monitored for 5 months using the following endpoints:
Biomicroscopy (slit lamp), including possible blood leakages
Endothelial swelling
Cloudiness and other symptoms in the anterior part of the eye
Lens opacity
Location/mobility of the implant and its possible fragmentation, photo of implant inside living eye
ERG (electroretinography), 2 . . . 3 times per study period
Intraocular pressure
Histology (in the end)

Results: There were no adverse effects caused by the implant compared to normal eye. Example picture of remaining implant in the Rabbit eye vitreous is depicted in the FIG. 29.

The following embodiments are typical of the present technology:

1. A composition comprising at least one active pharmaceutical ingredient, at least one biocompatible polymer and at least one biocompatible solvent, wherein
the composition is in the form of an injectable solution, suspension, emulsion or dispersion,
the composition is in the form of an in-situ forming implant composition, and the biocompatible polymer is bioresorbable.

2. The composition of embodiment 1, wherein the composition is suitable for treatment of glaucoma, intraocular pressure, wet Age-Related Macular Degeneration, dry Age-Related Macular Degeneration, diabetic retinopathy, dry eye syndrome, cataract, uveitis.

3, The composition of embodiment 1 or 2, wherein the active pharmaceutical ingredient is selected from active pharmaceutical ingredients for ophthalmic care.

4. The composition of any of embodiments 1 to 3, wherein the active pharmaceutical ingredient is selected from anti-VEGFs, and VEGFs (and their biosimilars), Tyrosine kinase inhibitors, antiparasites, H2 receptor antagonists, antimuscarinics, prostaglandins and its analogues, non-steroidal anti-inflammatory agents, proton pump inhibitors, am inosalycilates, corticosteroids, chelating agents, cardiac glycosides, phosphodiesterase inhibitors, thiazide, diuretics, anesthetic agents, carbonic anhydrase inhibitors, antihypertensives, anti-cancers, anti-depressants, calcium channel blockers, analgesics, opioid antagonists, antiplatels, anticoagulants, fibrinolytics, statins, adrenoceptor agonists, beta blockers, antihistamines, respiratory stimulants, micolytics, expectorants, barbiturates, anxiolytics, central nervous system agents, tricyclic antidepressants, 5HT1 antagonists, opiates, 5HT1 agonists, antiemetics, antiepileptics, dopaminergics, antibiotics, antifungals, anthelmintics, antivirals, antiprotozoals, antidiabetics, insulin and its derivatives, GLP-1 receptor agonists, thyrotoxins, antioestrogens, hypothalamics, pituitary hormones, posterior pituitary hormone antagonists, peptide drugs, protein drugs, protein kinases, antigens, antidiuretic hormone antagonists, bisphosphonates, dopamine receptor stimulants, androgens, steroid reductase inhibitors, non-steroidal anti-inflammatories, immunosuppressants, local anaesthetic, sedatives, anti-psoriatics, silver salts, topical antibacterials, vaccines, and vaccine antigens and combinations thereof.

5. The composition according to any of the preceding embodiment, wherein the active pharmaceutical ingredient is selected from Latanoprost, Tafluprost, Travoprost, Bimatoprost, Bevacizumab, Ranibizumab, Aflibercept, Adalimumab, Sunitinib, Axitinib, Regorafenib, Pazopanib, Dexamethasone, Fluocinolone, Cyclosporine, triamcinolone acetonide and combinations thereof.

6. The composition according to any of the preceding embodiment, wherein the bioresorbable polymer is selected from Polylactide (i.e. poly(lactic acid), PLA), polyglycolide (PGA) and poly(∈-caprolactone) (PCL), polydioxanone (PDO), polytrimethylenecarbonate (PTC), polylactides (PLA), poly-L-lactide (PLLA), poly-DL-lactide(PDLLA), PolyL-DL-lactide (PLDL), stereocopolymers; polyglycolide (PGA); copolymers of glycolide, glycolide/trimethylene carbonate copolymers (PGA/TMC); other copolymers of PLA, such as lactide/tetramethylglycolide copolymers, lactide/trimethylene carbonate copolymers, lactide/d-valerolactone copolymers, lactide/∈-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/∈-caprolactone terpolymers, PLA/polyethylene oxide copolymers; polydepsipeptides; unsymmetrically3,6-substitutedpoly-1,4-dioxane-2,5-diones; polyhydroxyalkanoates, such as polyhydroxybutyrates (PHB); PHB/b-hydroxyvalerate copolymers (PHB/PHV); poly-b-hydroxypropionate (PHPA); poly-p-dioxanone (PDS); poly-d-valerolactone-poly-∈-caprolactone, poly(∈-caprolactone-DL-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PVA); polypeptides; poly-b-malic acid (PMLA); poly-b-alkanoic acids; polycarbonates; polyorthoesters; polyphosphates; poly(ester anhydrides); and mixtures thereof; and natural polymers, such as sugars, starch, cellulose and cellulose derivatives, polysaccharides, collagen, chitosan, fibrin, hyaluronic acid, polypeptides and proteins or blends and combinations thereof.

7. The composition according to any of the preceding embodiments, wherein the polyester is non-linear, such as branched (including star-shaped or hyperbranched or dendrimers).

8. The composition according to any of the preceding embodiments, wherein the bioresorbable polymer is a purified polyester, wherein the metal content of the polyester is <40 ppm and the metal selected from tin, zinc, iron, aluminium, titanium, platinum, bismuth, manganese, antimony, nickel, calcium, magnesium, sodium, lithium, yttrium, lanthanum, samarium, zirconium, ruthenium and combinations thereof.

9. The composition according to any of the preceding embodiments, wherein the dynamic viscosity, at 25° C., of the composition is between 10 and 100,000 mPas, preferably 100 to 3000 mPas and more preferably 200 to 2000 mPas, measured with a rheometer and extrapolating the shear rate to 0 5-1.

10. The composition according to any of the preceding embodiments, which composition is formulated for use with an injection needle having a diameter corresponding to any of classes G20 to G31 or thinner, typically G26 to G31.

11. A controlled release pharmaceutical composition for eye injection comprising a composition according to any of embodiments 1 to 10 for use in therapy of ophthalmic conditions.

12. The pharmaceutical composition for use according to embodiment 11, wherein the injection is given to any part of the eye comprising intravitreally, intracamerally, periocularly, subchoroidally, subconjunctivally, subretinal, or under eye mucosal layer or utilizing pre-injected watery bleb under the surface of the eye.

13. The pharmaceutical composition for use according to embodiment 12 or 13, wherein the injection is given to the eye as needed or regularly between once a week and annually.

### REFERENCE NUMERALS

1 Epidermis
2 SC fatty tissue
3 Muscle
4, 4a, 4b ISFI
5, 5a Syringe
6 Eye
11 Cap
12 Needle
13 Syringe
14 Syringe A
Syringe B
16 Coupler
17 Cap
18 Needle
19 Mixing
Ready-to-use syringe
21 Plunger
22 Piston 1
23 Piston 2
24 Septum
Needle

1-27. (canceled)

28. A controlled release pharmaceutical composition for eye injection for ophthalmic care, comprising:
at least one active pharmaceutical ingredient and at least one biocompatible polymer and at least one biocompatible solvent, wherein:
i) the controlled release pharmaceutical composition is in the form of an injectable solution, suspension, emulsion or dispersion;
ii) the composition is in the form of an in-situ forming implant composition; and
iii) the at least one biocompatible polymer is bioresorbable,
iv) the bioresorbable polymer comprises a polyester,
iv) the composition has a volume of from 1 microliter to 100 microliter, and
v) wherein the metal content of the polyester is <40 ppm

29. The controlled release pharmaceutical composition according to claim 28, wherein the metal is selected from the group consisting of tin, zinc, iron, aluminium, titanium, platinum, bismuth, manganese, antimony, nickel, calcium, magnesium, sodium, lithium, yttrium, lanthanum, samarium, zirconium, ruthenium, and combinations thereof.

30. The controlled release pharmaceutical composition according to claim 28, wherein the active pharmaceutical ingredient is selected from the group consisting of anti-VEGFs, VEGFs and their biosimilars, Tyrosine kinase inhibitors, antiparasites, H2 receptor antagonists, antimuscarinics, prostaglandins and its analogues, non-steroidal anti-inflammatory agents, proton pump inhibitors, aminosalycilates, corticosteroids, chelating agents, cardiac glycosides, phosphodiesterase inhibitors, thiazide, diuretics, anesthetic agents, carbonic anhydrase inhibitors, antihypertensives, anti-cancers, anti-depressants, calcium channel blockers, analgesics, opioid antagonists, antiplatels, anticoagulants, fibrinolytics, statins, adrenoceptor agonists, beta blockers, antihistamines, respiratory stimulants, micolytics, expectorants, barbiturates, anxiolytics, central nervous system agents, tricyclic antidepressants, 5HT1 antagonists, opiates, 5HT1 agonists, antiemetics, antiepileptics, dopaminergics, antibiotics, antifungals, anthelmintics, antivirals, antiprotozoals, antidiabetics, insulin and its derivatives, GLP-1 receptor agonists, thyrotoxins, antioestrogens, hypothalamics, pituitary hormones, posterior pituitary hormone antagonists, peptide drugs, protein drugs, protein kinases, antigens, antidiuretic hormone antagonists, bisphosphonates, dopamine receptor stimulants, androgens, steroid reductase inhibitors, non-steroidal anti-inflammatories, immunosuppressants, local anaesthetic, sedatives, anti-psoriatics, silver salts, topical antibacterials, vaccines, vaccine antigens, and combinations thereof.

31. The controlled release pharmaceutical composition according to claim 28, wherein the active pharmaceutical ingredient is selected from the group consisting of Latanoprost, Tafluprost, Travoprost, Bimatoprost, Bevacizumab, Ranibizumab, Aflibercept, Adalimumab, Sunitinib, Axitinib, Regorafenib, Pazopanib, Dexamethasone, Fluocinolone, Cyclosporine, triamcinolone acetonide, and combinations thereof.

32. The controlled release pharmaceutical composition according to claim 28, wherein the polyester is purified, and wherein the purification comprises the steps of:
i) dissolving the polyester in an organic solvent to form a polyester-solvent solution, wherein the organic solvent comprises at least one heteroatom selected from oxygen, nitrogen, sulphur, chlorine and phosphorus;
ii) precipitating the polyester from the polyester-solvent solution by combining the polyester-solvent solution with an organic non-solvent, said non-solvent being an alcohol; and
(iii) separating the precipitated polyester from the solvent and non-solvent to obtain a purified polyester.

33. The controlled release pharmaceutical composition according to claim 28, wherein the total amount of residual monomers in the composition is less than 0.2 wt %.

34. The controlled release pharmaceutical composition according to claim 28, wherein the polyester is selected from the group consisting of polylactic acid, polyglycolic acid, polycaprolactone, poly(lactide-co-glycolide), poly(lactide-co-caprolactone), poly(glycolide-co-caprolactone), and poly(lactide-co-glycolide-co-caprolactone).

35. The controlled release pharmaceutical composition according to claim 28, wherein the polyester is a poly(lactide-co-glycolide).

36. The controlled release pharmaceutical composition according to claim 28, wherein the polyester is a purified polyester.

37. The controlled release pharmaceutical composition according to claim 28, wherein the polyester is non-linear, or branched.

38. The controlled release pharmaceutical composition according to claim 28, wherein the at least one biocompatible solvent is capable of dissolving at least 1 mg/ml of the polyester at 35-37° C., and is capable of dissolving, dispersing or suspending the at least one active pharmaceutical ingredient.

39. The controlled release pharmaceutical composition according to claim 28, wherein the composition comprises at least one biocompatible solvent selected from the group consisting of N-methyl-2-pyrrolidone, triacetin, dimethylsulfoxide, benzyl benzoate, benzyl alcohol, triethyl citrate, triethyl acetyl citrate, ethyl acetate, anisole, glycofurol, PEG (polyethylene glycol), PPG (polypropylene glycol), polycaprolactones, and combinations thereof.

40. The controlled release pharmaceutical composition according to claim 28, wherein the composition further comprises a biocompatible excipient selected from the group consisting of vanillin, SAIB, polycaprolactonediol, hydroxypropylmethylcellulose, HP-b-CD, cyclodextrins, MCT, dextrans, sucrose, crown ethers, chitosan, mannitol, trehalose, and combinations thereof.

41. The controlled release pharmaceutical composition according to claim 28, wherein the weight ratio between the at least one biocompatible polymer and the at least one biocompatible solvent is in the range from 10:90 to 90:10.

42. The controlled release pharmaceutical composition according to claim 28, wherein the controlled release pharmaceutical composition has a volume of between 1 microliter and 50 microliter.

43. The controlled release pharmaceutical composition according to claim 28, wherein the loading of the at least one active pharmaceutical ingredient in the composition is between 0.1 wt % and 90 wt % of the total weight of the at least one biocompatible polymer and the at least one biocompatible solvent.

44. The controlled release pharmaceutical composition according to claim 28, wherein the dynamic viscosity, at 25° C., of the composition is between 10 and 100,000 mPas.

45. The controlled release pharmaceutical composition according to claim 28, formulated for delivery by an injection needle having a diameter corresponding to any of classes G20 to G31.

46. A drug product for eye injection comprising:
i) a closed syringe comprising the controlled release pharmaceutical composition of claim 1 and a needle for injection; or
ii) a closed vial or ampule containing the controlled release pharmaceutical composition of claim 1, an empty syringe to be filled from the vial or ampule containing the controlled release pharmaceutical composition, and a needle for injection, or
iii) a closed syringe comprising the controlled release pharmaceutical composition of claim 28, an empty syringe to be filled from the closed syringe, and a needle for injection.

47. A drug product for eye injection comprising:
a kit containing a first syringe with a placebo formulation and a second syringe with the controlled release pharmaceutical composition of claim 28, the first and second syringes connected to each other and the contents of each mixed with each other to form a mixture upon reciprocal action between the syringes; and
a needle attached to the syringe where the mixture is left.

48. A method of treating a subject in need of ophthalmic care, the method comprising administering to the eye of said subject an injection of the controlled release pharmaceutical composition according to claim 28.

49. The method according to claim 48, wherein the injection is given to any part of the eye intravitreally, intracamerally, periocularly, subchoroidally, subconjunctivally, subretinal, under an eye mucosal layer, or utilizing a pre-injected watery bleb under the surface of the eye.

50. The method according to claim 48, wherein the injection is given to the eye intravitreally or subconjunctivally and has a volume of 1 to 50 μl.

51. The method according to claim 48, wherein the injection is administered to the eye of the subject as needed or regularly between once a week and annually.

52. The method according to claim 48, wherein the controlled release pharmaceutical composition is administered for the treatment of a member selected from the group consisting of glaucoma, intraocular pressure, wet Age-Related Macular Degeneration, dry Age-Related Macular Degeneration, diabetic retinopathy, dry eye syndrome, cataract, and uveitis.
